(19) United States
(12) Patent Application Publication
(10) Pub. No.: US 2005/0038428 A1
(43) Pub. Date: Feb. 17, 2005

(54) Methods and devices for retaining cerclage cable

(76) Inventors: David C. Kelman, Collierville, TN (US); C. Wayne Allen, Southaven, MS (US)

Correspondence Address: CHIEF PATENT COUNSEL;SMITH & NEPHEW, INC., 1450 BROOKS ROAD, MEMPHIS, TN, 38116, US

(21) Appl. No.: 10/903,400

(22) Filed: Jul. 30, 2004

Related U.S. Application Data
(60) Provisional application No. 60/491,071, filed on Jul. 30, 2003.

(51) Int. Cl. A61F 5/04; A61B 17/56; A61F 2/30

(52) U.S. Cl. 606/60; 606/53; 606/74

(57) ABSTRACT

Methods and devices for retaining cerclage cable are described. According to one embodiment, a method comprising initiating a surgical procedure wherein an orthopedic device is secured to a bone at least in part with cerclage cable, and wherein the orthopedic device comprises an opening configured to receive a retaining device, providing a retaining device, wherein the retaining devices comprises a shaft, the shaft comprising a proximal end, the proximal end comprising a head containing retaining structure configured to secure the cerclage cable, and whereby the cerclage cable does not thread through an opening in the retaining device, and wherein the shaft further comprises a distal end configured to be inserted through the orthopedic device into the bone, inserting the retaining device through the opening in the orthopedic device and into the bone, securing the cerclage cable in the retaining structure in the proximal end of the retaining device, and completing the surgical procedure with the orthopedic device, cerclage cable, and retaining device in place is described.

### RELATED APPLICATIONS

This application claims the benefit of the U.S. provisional application, application No. 60/491,071, entitled “Methods and Apparatus for Retaining a Cerclage Cable” filed Jul. 30, 2003, which is hereby incorporated by this reference.

### FIELD OF THE INVENTION

The invention relates generally to devices and methods for use in orthopedic procedures. More particularly the invention relates to methods and devices for retaining cerclage cable.

### BACKGROUND OF THE INVENTION

Surgical cerclage cable is used in a variety of surgical procedures including total hip arthroplasty and fracture fixation. In these surgical procedures the cerclage cable may be used in combination with a bone plate or trochanteric grip to help secure and stabilize a bone, such as the femur, and other anatomical or surgical structure, in a proper orientation and alignment. Additionally, fractures involving large bones such as the femur can be difficult to immobilize, and often require the use of bone screws, cables and/or bone plates to securely reunite fractured bone segments.

One frequently used procedure involves wiring the fractured bone to a rod or plate that is attached at multiple attachment points along the femur or other bony structure on either side of a fracture. The plate can be fastened to the bone using bone screws or bone spikes and the attachment can be reinforced by encircling both the bone and the bone plate with cerclage cable.

Traditionally, cerclage cable is looped around the bone and bone plate and can then be secured in position on the bone plate by threading the cable through an opening in a structure attached to the plate. The loop formed by the cerclage cable can then be tightened and the cerclage cable can be secured to itself by means of a deformation crimping device in order to maintain tension in the loop and prevent undesired movement or shifting of the cerclage cable or the underlying anatomical or surgical structure.

By way of example, screw fixation is frequently not adequate for fractures that are highly comminuted, and fixation with cables, or cables in combination with plates, is required. Cables are also sometimes used in conjunction with screws, and in conjunction with screws in combination with plates. By way of example, cables may be used in conjunction with screws, and screws in combination with plates, where a screw strips its threads in deployment, or where screws, or screws in combination with plates, is deemed insufficient for the fixation at hand.

With cable fixation, it is customary to encircle the subject bone with the body of the cable, thread the cerclage cable through a retaining device, and then to secure the two free ends of the cable together with a deformable crimp so as to effect fixation. In general, the difficultly of cable fixation on the proximal or distal tip of a bone can be enhanced due to a significant flare in the geometry of the distal or proximal tip. For example, securing a trochanteric grip on the proximal end of the femur with cerclage cable is complicated by the geometry of the intertrochanteric crest, and the flare of the greater trochanter. As a result, tightening of the encircling cable in such a situation often causes the cable (and crimp) to migrate toward a narrower section of the bone. Such migration can displace the cable sufficiently far from its intended location as to significantly loosen, or render ineffective, encirclement of the bone segments.

Additionally, traditional cable fixation techniques often require threading the cerclage cable through a retaining device. This requires that one end of the cerclage cable be fed through the retaining device prior to tightening and crimping the cerclage cable. Threading the cable through a retaining device in the limited space of a surgical site can be difficult and can present additional chance of error. Also, such retaining devices do not allow for easy reorientation once the cable is threaded through the device and the cerclage cable is wrapped around the surgical or anatomical structures.

Moreover, traditional methods involving bone screws to assist in securing a surgical structure typically require an additional device for retaining the cerclage cable in proper alignment. This increases instrument count and increases a likelihood of error. Also, bone screws can exhibit a tendency to loosen up after being used to secure a bone plate or other structure to a bone.

Thus, there is a need for improved methods and devices that will permit bone fixation to be effected with screws and cable such that the cable cannot migrate along the bone after fixation, such that additional retaining devices are not required, such that the orientation of the retaining device can be more easily adjusted throughout the procedure, such that there is no need to thread the cerclage cable through an opening in the retaining device, such that the cable can efficiently be placed into and removed from the retaining device, and such that the retaining method can help secure the bone screws in proper position.

### SUMMARY OF THE INVENTION

Various embodiments of the present invention provide methods and devices for retaining cerclage cable. According to one embodiment of the present invention, a method comprises initiating a surgical procedure wherein an orthopedic device is secured to a bone at least in part with cerclage cable, and wherein the orthopedic device comprises an opening configured to receive a retaining device, providing a retaining device, wherein the retaining devices comprises a shaft, the shaft comprising a proximal end, the proximal end comprising a head containing retaining structure configured to secure the cerclage cable, and whereby the cerclage cable does not thread through an opening in the retaining device, and wherein the shaft further comprises a distal end configured to be inserted through the orthopedic device into the bone, inserting the retaining device through the opening in the orthopedic device and into the bone, securing the cerclage cable in the retaining structure in the proximal end of the retaining device, and completing the surgical procedure with the orthopedic device, cerclage cable, and retaining device in place.

According to another embodiment, the retaining structure can comprise at least one slot perpendicular to a central axis of the shaft extending across the head. According to another embodiment the retaining structure can comprise at least one notch extending partially across the head. According to another embodiment the shaft of the retaining device can comprise a bone screw or bone spike and the spike can comprise a conical shaft with a distal end, wherein the distal end forms a point, the conical shaft comprising projections configured to secure the bone spike in a desired position upon insertion of the spike into a bone. According to another embodiment the head of the retaining device can be configured to engage a tool used to drive the bone screw into the bone. According to another embodiment the head of the retaining device can comprise a hex socket. According to another embodiment the orthopedic device can comprise a bone plate.

### BRIEF DESCRIPTION OF THE DRAWINGS

FIG. 1 illustrates a device for retaining cerclage cable according to one embodiment of the present invention;

FIG. 2 illustrates another device for retaining cerclage cable according to one embodiment of the present invention;

FIG. 3 illustrates a device for retaining cerclage cable according to one embodiment of the present invention in use with a bone plate attached to a bone with cerclage cable;

FIG. 4 is a top view of a portion of the device illustrated in FIG. 3; and

FIG. 5 illustrates a method for retaining cerclage cable according to one embodiment of the present invention.

### DETAILED DESCRIPTION

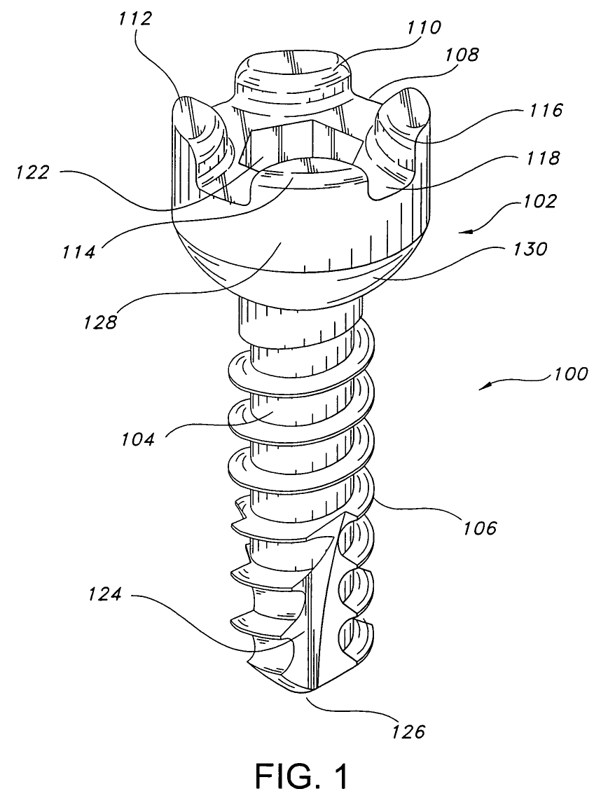

Methods and devices according to certain embodiments of the present invention assist a surgeon position and retain cerclage cable during a surgical procedure. According to one embodiment, a retaining device 100 is provided. According to the embodiment depicted in FIG. 1, the retaining device 100 comprises a shaft 104. The shaft 104 comprises a proximal end and a distal end. The proximal end of the shaft 104 forms a head 102 and the distal end of the shaft 104 forms a tip 126. The head 102 comprises a cylindrical surface 128 and a substantially spherical surface 130. The substantially spherical surface 130 joins the proximal end of the shaft to the cylindrical surface 128. The spherical surface 130 is configured to rotate and pivot smoothly within a receiving opening in a bone plate or other suitable orthopedic device.

A distal portion of the cylindrical surface 128 joins with a proximal portion of the substantially spherical surface 130. The cylindrical surface 128 continues proximally from the substantially spherical surface 130 to terminate in raised portions 110-116 defining retaining structure 108 on a proximal end of the head.

The retaining structure 108 is configured such that cerclage cable can be retained by the retaining structure 108 without threading the cerclage cable through an opening. For example, according to the embodiment depicted in FIG. 1, the retaining structure 108 is a slot formed by the raised portions 110-116. The slot is configured such that a cerclage cable can be placed into the opening defined by the raised portions 110-116 and is retained within the slot 108 by the raised portions 110-116 as to prevent slippage of the cerclage cable. During use, the cable can be placed into the slot 108 and tension can be applied to further secure the cerclage cable in position.

While the retaining structure 108 is depicted in FIG. 1 as a slot formed by the raised portions 110-116, according to another embodiment, the retaining structure can comprise a notch or other suitable structure for retaining cerclage cable. For example, the notch can comprise a raised portion extending partially across the diameter of the head 102. The notch can comprise, for example, a raised portion of the head 102 with a flange or other suitable retaining mechanism designed to receive and secure cerclage cable wherein the cerclage cable is not threaded through an opening in the retaining device.

Additionally, according to one embodiment the head 102 may comprise only a single retaining structure. According to another embodiment, the head 102 may comprise more than two retaining structures. The retaining structure 100 illustrated in FIG. 1 comprises two slot retaining structures 108 and 118 formed by the raised structures 110-116 and is offered as only one embodiment of a retaining structure that may be used in the present invention.

The head 102 can be configured to facilitate engagement of a driving device for driving the retaining device 100 into position. According to the embodiment depicted in FIG. 1, the head comprises a hex socket recess 122 designed to engage a hex-shaped driving device. According to other embodiments other shapes of the recess 122 may be employed. Additionally, other suitable structure or methods for facilitating engagement of a driving device with the retaining device 100 may be used as part of the present invention.

The shaft 104, according to the embodiment depicted in FIG. 1, comprises threads 106. The threads 106 encircle the shaft 104 and come to a tip 126 forming a bone screw. The shaft 104 may further comprise a groove 124 located at the distal end of the shaft 104 to facilitate insertion into a bone. According to another embodiment of the present invention, the shaft may employ structure other than threads for insertion into a bone.

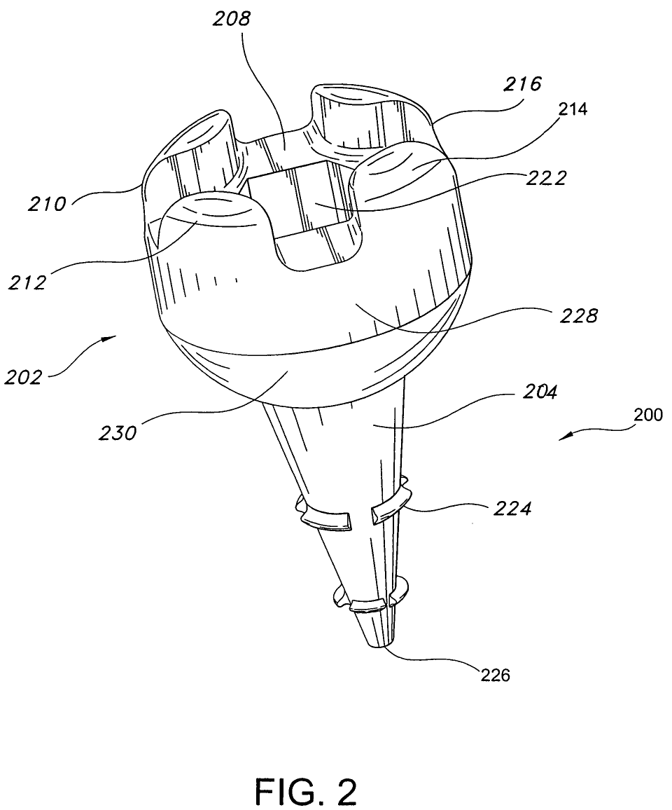

For example, FIG. 2 illustrates one embodiment of the present invention, wherein a shaft 204 is substantially conical in shape forming a point 226 at a distal end of the shaft. The conical shaped shaft 204 thus forms a spike for insertion into a bone and can further comprise projections 224 for facilitating fixation into the bone. The projections 224 may be oriented either longitudinally or transversely with respect to a central axis of the shaft 204. For purposes of illustration, the projections 224 illustrated in FIG. 2 are oriented transversely with respect to the central axis of the shaft 204. The geometry of the projections 224 may be curved to allow the shaft 204 to be easily inserted into a bone but to catch or grab the bone upon motion tending to back the shaft 204 out of the bone as to inhibit dislocation of the shaft 204.

According to another embodiment, the head 102 of the retaining device 100 may be used without a shaft 104. For example, the head 102 can be configured to be received into an opening in a bone plate or other orthopedic device configured to receive a bone screw. Thus, according to one embodiment, just a head portion 102 comprising the spherical surface 130 can be inserted into an opening in a bone plate. Cerclage cable can then be secured in the retaining structure 108 of the head 102. The tension from the cerclage cable can prevent movement of the head 102 without requiring the shaft 104 to be inserted into the bone, and the head 102 can prevent slippage of the cerclage cable by retaining the cable in the retaining structure 108.

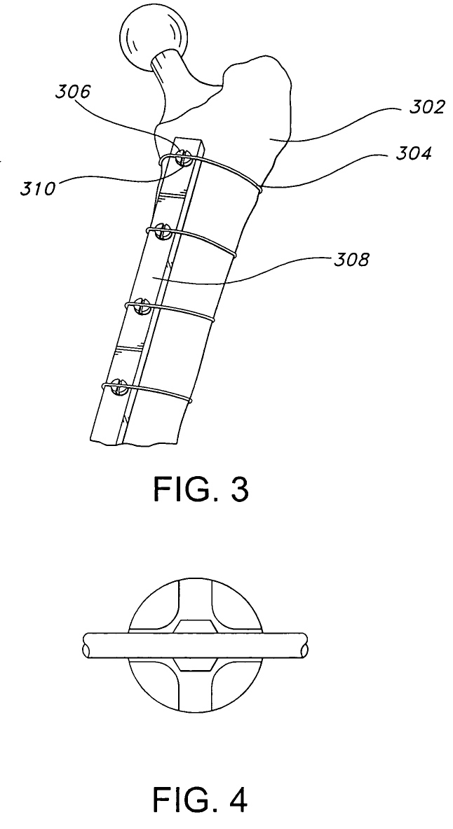

FIG. 3 illustrates one embodiment of the present invention in use with a bone plate 308, cerclage cable 304, femur 302, and retaining device 306. The retaining device 306 illustrated in FIG. 3 comprises a retaining device similar to the device illustrated in FIG. 1 or FIG. 2. In FIG. 3, only the head of the retaining device 306 is visible.

In use, the retaining device 306 is inserted through an opening 310 in the bone plate 308 and into the bone 302. According to one embodiment, the opening 310 is configured to allow a substantially spherical portion of a head of the retaining device 306 to pivot and rotate smoothly. Cerclage cable 304 is wrapped around the bone 302 and the bone plate 308 and placed in the retaining structure on the head of the retaining device 306. FIG. 4 illustrates a close-up view of the top of the retaining structure on the retaining device 306. As illustrated in FIG. 4, the cerclage cable can be placed into the slot formed by the raised portions of the retaining structure. Once the cerclage cable is in place, the cable can be tightened according to standard procedure. It will be appreciated that the tension on the bone screw helps immobilize and prevent undesired movement of the bone screw, and the retaining structure prevents the cerclage cable from slipping relative to the bone plate.

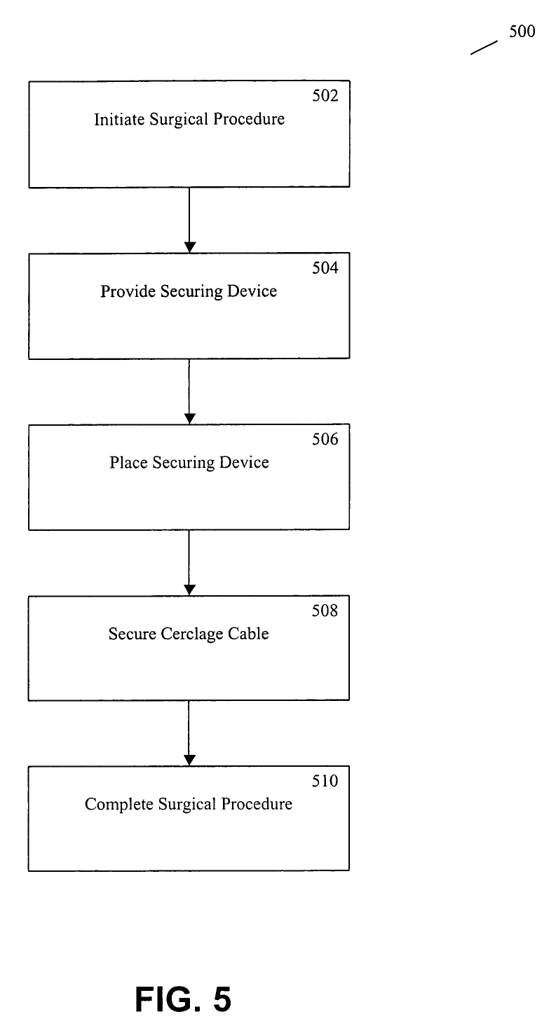

A method 500 for securing cerclage cable during a surgical operation according to one embodiment of the present invention is illustrated in FIG. 5. According to the embodiment illustrated in FIG. 5, the method 500 begins in block 502, wherein a surgical procedure in which an orthopedic device is to be secured with cerclage cable is initiated according to standard surgical techniques. The procedure initiated in block 502 can comprise, for example, a procedure for fracture fixation using a bone plate, a procedure for securing a hip implant component using a trochanteric grip, or other suitable procedure in which an orthopedic device is to be secured with cerclage cable.

Once the procedure is initiated in block 502, the method 500 proceeds to block 504, wherein a retaining device is provided. The retaining device provided in block 504 comprises a retaining device as discussed above in connection with FIGS. 1-4 comprising a shaft and a head formed at the proximal end of the shaft, the head comprising retaining structure configured to secure cerclage cable without threading an end of the cerclage cable through an opening in the retaining device.

Once a retaining device is provided in block 504, the method 500 proceeds to block 506, wherein the retaining device is placed into position. Placing the retaining device into position can comprise inserting the retaining device into an opening in the orthopedic device, such as the openings 310 illustrated in FIG. 3, and driving the device into the bone. According to one embodiment, driving the device into the bone can be accomplished by twisting the retaining device with a driving means configured to engage a recess in the head of the retaining device such as the hex socket recess 122 illustrated in FIG. 1.

Once the device is placed into position, the method 500 proceeds to block 508, wherein cerclage cable is secured in place. Securing the cerclage cable can comprise wrapping the cable around the orthopedic device and the bone and then placing the cerclage cable into a slot or other suitable retaining structure on the retaining device. In order to secure the cerclage cable, it is necessary for the retaining structure in the head of the retaining device to align with the cable. It will be appreciated that embodiments of the present invention allow for adjusting the orientation of the retaining structure simply by rotating, pivoting, or twisting the head of the retaining device and that the substantially spherical portion of the head can facilitate such movement. This allows for easy alignment of the retaining structure and efficient placement of the cerclage cable.

It will also be appreciated that the present invention allows for placement of the cerclage cable into the retaining device after the cable ends have been joined together and the cable has been wrapped around the orthopedic device and the bone, and that this can be accomplished without the need to thread the cable through an opening. The present invention allows the cerclage cable to simply be laid into position and then tightened according to standard procedure. Once the cable is tightened, the tension on the cable together with the retaining structure in the head of the retaining device will prevent slippage or undesired movement of the cable.

It will further be appreciated that because the cerclage cable and the retaining device are not mechanically linked, that the cerclage cable can be moved by simply releasing the tension on the cable without the need to unthread the cable.

Following securing of the cerclage cable in block 508, the method 500 proceeds to block 510, wherein the surgical procedure is complete according to standard surgical techniques with the cerclage cable, retaining device, and orthopedic device in place.

The foregoing has been provided for purposes of disclosure of preferred embodiments of the invention. Changes, additions, omissions may be made to the devices and processes disclosed in this document without departing from the scope or spirit of the invention.

1. A method of securing an orthopedic device using cerclage cable, comprising:
initiating a surgical procedure wherein an orthopedic device is secured to a bone at least in part with cerclage cable, and wherein the orthopedic device comprises an opening configured to receive a retaining device;
providing a retaining device, wherein the retaining devices comprises a shaft, the shaft comprising a proximal end, the proximal end comprising a head containing retaining structure configured to secure the cerclage cable, and whereby the cerclage cable does not thread through an opening in the retaining device, and wherein the shaft further comprises a distal end configured to be inserted through the orthopedic device into a bone;
inserting the retaining device through the opening in the orthopedic device and into the bone;
securing the cerclage cable in the retaining structure in the proximal end of the retaining device; and
completing the surgical procedure with the orthopedic device, cerclage cable, and retaining device in place.

2. The method of claim 1, wherein the retaining structure comprises at least one slot perpendicular to a central axis of the shaft extending across the head.

3. The method of claim 1, wherein the retaining structure comprises at least one notch extending partially across the head.

4. The method of claim 1, wherein the shaft of the retaining device comprises a bone screw.

5. The method of claim 1, wherein the head of the retaining device is configured to engage a tool used to drive the shaft into the bone.

6. The method of claim 5, wherein the head of the retaining device comprises a driver socket.

7. The method of claim 1, wherein the shaft comprises a spike.

8. The method of claim 7, wherein the spike comprises a conical shaft with a distal end, wherein the distal end forms a point, the conical shaft comprising projections configured to secure the bone spike in a desired position upon insertion of the spike into a bone.

9. The method of claim 1, wherein the orthopedic device comprises a bone plate.

10. A system for retaining cerclage cable, comprising:
cerclage cable;
an orthopedic device configured to receive a retaining device; and
a retaining device comprising a shaft wherein the shaft comprises a proximal end and a distal end; the proximal end comprising a head containing at least one slot configured to secure cerclage cable whereby the cerclage cable does not thread through an opening in the retaining device; the distal end configured to be inserted through the orthopedic device and into the bone;

11. The system of claim 10, wherein the retaining structure comprises at least one slot perpendicular to a central axis of the shaft extending across the head.

12. The system of claim 10, wherein the retaining structure comprises at least one notch extending partially across the head.

13. The system of claim 10, wherein the shaft of the retaining device comprises a bone screw.

14. The system of claim 10, wherein the head of the retaining device is configured to engage a tool used to drive the shaft into the bone.

15. The system of claim 14, wherein the head of the retaining device comprises a driver socket.

16. The system of claim 10, wherein the shaft comprises a spike.

17. The system of claim 16, wherein the spike comprises a conical shaft with a distal end, wherein the distal end forms a point, the conical shaft comprising projections configured to secure the bone spike in a desired position upon insertion of the spike into a bone.

18. The system of claim 10, wherein the orthopedic device comprises a bone plate.

19. A method for securing cerclage cable comprising:
initiating a surgical procedure wherein an orthopedic device is secured to a bone at least in part with cerclage cable, and wherein the orthopedic device comprises an opening configured to receive a retaining device;
providing a retaining device, wherein the retaining devices comprises a circular member containing at least one slot configured to secure cerclage cable whereby the cerclage cable does not thread through an opening on the circular member; the circular member configured to fit within an opening of an orthopedic device; whereby the circular member rotates freely to align with the cerclage cable; and whereby the circular member remains fixed upon receiving the cerclage cable and the cerclage cable being tightened;
inserting the retaining device into the opening in the orthopedic device;
securing the cerclage cable in the retaining structure; and
completing the surgical procedure with the orthopedic device, cerclage cable, and retaining device in place.

20. The method of claim 19, wherein the retaining structure comprises at least one slot extending across the head.

21. The method of claim 19, wherein the retaining structure comprises at least one notch extending partially across the head.

22. The method of claim 19, wherein the orthopedic device comprises a bone plate.

23. A system for retaining cerclage cable, comprising:
cerclage cable;
an orthopedic device configure to receive a retaining device; and
a retaining device comprising a circular member containing at least one slot configured to secure cerclage cable whereby the cerclage cable does not thread through an opening on the circular member; the circular member configured to fit within an opening of an orthopedic device; whereby the circular member rotates freely to align with the cerclage cable; and whereby the circular member remains fixed upon receiving the cerclage cable and the cerclage cable being tightened.

24. The system of claim 23, wherein the retaining structure comprises at least one slot extending across the head.

25. The system of claim 23, wherein the retaining structure comprises at least one notch extending partially across the head.

26. The system of claim 23, wherein the orthopedic device comprises a bone plate.
